(19) United States
(12) Patent Application Publication
(10) Pub. No.: US 2004/0137541 A1
(43) Pub. Date: Jul. 15, 2004

(54) Method for detecting cancer associated with elevated concentrations of lysophospholipids

(76) Inventors: Gordon B. Mills, Houston, TX (US); Bruce John Holub, Guelph (CA); Douglas Craig Gaudette, Guelph (CA)

Correspondence Address: Baker & Hostetler LLP, Washington Square, Suite 1100, 1050 Connecticut Avenue N.W., Washington, DC, 20036, US

(21) Appl. No.: 10/701,558

(22) Filed: Nov. 6, 2003

Related U.S. Application Data
(63) Continuation of application No. 08/822,128, filed on Mar. 21, 1997, now abandoned.

(51) Int. Cl. G01N 33/574; G01N 33/48

(52) U.S. Cl. 435/7.23; 436/64

(57) ABSTRACT

The present invention is methods for detecting the presence of cancer in a subject by determining the concentrations of lysophospholipids in a sample of bodily fluid taken from a test subject and comparing these concentrations to concentrations present in samples taken from normal subjects without cancer. The methods may be used for diagnosis and prognosis of cancer in a subject and to monitor the results of therapy of over time.

### 1. INTRODUCTION

The present invention relates to methods for screening subjects for the presence of cancers, particularly gynecological cancer, correlated with elevated concentrations of lysophospholipids, by detecting the concentration of the lysophospholipids in a sample of bodily fluid from a test subject.

### 2. BACKGROUND OF THE INVENTION

There is an ongoing need for cancer markers for early detection of a variety of cancers in humans. Certain cancers, such as gynecological cancers, present a serious mortality factor for women and pose a challenge for early intervention. For example, ovarian cancer is the fourth leading cause of death from malignancy in women (American Cancer Society, Cancer J. Clin., 43:7-26 (1993)). Diagnosis at an early, more treatable stage could bring about higher survival rates in ovarian cancer where 70% of patients currently present with advanced disease at the time of diagnosis. The best available serum marker, CA125, does not have sufficient sensitivity or specificity to warrant use as a sole marker in screening for ovarian cancer (Einhorn et al., Obstet. Gynecol. 80:14-18 (1992)). In particular, CA125 is not detected in serum from up to 50% of patients with early-stage ovarian cancer (Schapira et al., Ann. Intern. Med., 118:83B-843 (1993)). No reliable plasma or serum markers exist for detecting cervical or uterine cancer. Moreover, the rate of false positive results for use of CD125 as a marker, approximately 2%, leads to approximately 100 false positive results for each early cancer detected. (Jacobs et al., BMJ, 313:(7069):1355-1358 (1996); Dorum et al., E. J. Cancer 32A(10):1645-1651 (1996); Muto et al., Gynecologic Oncol. 51(1):12-20 (1993); Jacobs et al., Lancet 1(8580):268-271 (1988)

More reliable markers for economically and rapidly screening subjects for early detection of cancers, particularly gynecological cancers, establishing the subject's prognosis and monitoring the subject's response to therapy of these cancers, are required to improve the prognosis of these diseases.

Phosphatidylcholine (PC) is one of the major sources of polyunsaturated fatty acids such as arachidonic and linoleic acids. The former is a precursor of eicosanoids which have numerous biological activities. Hydrolysis of PC yields lysophosphatidyl choline (LysoPC) and constituent fatty acids, which have been implicated in signal transduction (Asaoka et al., Proc. Natl. Acad. Sci. USA, 90:4917-4921 (1993); Yoshida et al., Proc. Natl. Acad. Sci. USA, 89:6443-6446 (1992)). An increasing body of evidence indicates that LysoPC, which is present in high concentrations in oxidized low density lipoproteins (for review see Steinberg et al., Eng. J. Med. 320:915-924 (1989)), may play a significant-role in atherogenesis and other inflammatory disorders. For example, LysoPC has been reported to increase the transcription of the genes encoding platelet derived growth factor A and B chains, and heparin-binding epidermal growth factor-like protein (HB-EGF) in cultured endothelial cells (Kume and Gimbrone, J. Clin. Invest. 93:907-911 (1994)), and to increase mRNA encoding HB-EGF in human monocytes (Nakano et al., Proc. Natl. Acad. Sci. USA 91:1069-1073 (1994)). Both of these gene products are potent mitogens for smooth muscle cells and fibroblasts (Higashiyama et al., Science 251:936-939 (1991); Ross, Nature (Lond.) 362:801-809 (1993)). LysoPC has also been reported to activate protein kinase C in vitro (Sasaki et al., FEBS Letter 320:47-51 (1993)), to potentiate the activation of human T lymphocytes (Asaoka et al., Proc. Natl. Acad. Sci. USA 89:6447-6451 (1992)), and to potentiate the differentiation of HL-60 cells to macrophages induced by either membrane-permeable diacylglycerols or phorbol esters (Asaoka et al., Proc. Natl. Acad. Sci. USA 90:4917-4921 (1993)).

LysoPC may also provide a source of bioactive lysophosphatidic acid (LPA) (for review see Moolenaar et al., Rev. Physiol. Biochem. Pharmacol. 119:47-65 (1992)) through hydrolysis by lysophospholipase D (Tokumura et al., Biochim. Biophys. Acta 875:31-38 (1986)). Ovarian cancer activating factor (OCAF), has been isolated from ovarian cancer ascites fluid (Mills et al., Cancer Res. 48:1066 (1988); Mills et al. J. Clin. Invest. 86:851 (1990) and U.S. Pat. Nos. 5,326,690 and 5,277,917) and has been identified to consist of multiple forms of LysoPA (Xu et al., Clin. Cancer Res. 1:1223-1232 (1995)). LysoPA has been identified as a potent tumor growth factor in the ascites fluid of ovarian cancer patients (Id.).

### 3. SUMMARY OF THE INVENTION

Accordingly, the present invention encompasses methods for diagnosing, determining the prognosis of and monitoring cancers, including gynecological cancers such as ovarian, uterine, fallopian tube and cervical cancers, correlated with elevated concentrations of certain lysophospholipids in a subject relative to the levels of lysophospholipids in normal subjects without cancer.

The method is carried out by detecting the concentration of a lysophospholipid in a sample of bodily fluid taken from a subject. This measurement may be taken as 1) the concentration of the specific lysophospholipid selected, e.g. LysoPC or LysoPA present in the sample from the subject; 2) the concentration of a subtype of the selected lysophospholipid having a particular degree of saturated or unsaturated fatty acids and/or fatty acid chain length (e.g. palmitoyl-LysoPC or linoleoyl-LysoPC) or the concentration of a subtype having a particular long chain alcohol attached to the glycerol backbone; 3) the concentration of total lysophospholipids present in a sample; or 4) the concentration of first one lysophospholipid, e.g., LysoPC, followed by measurement of another lysophospholipid, e.g. LysoPA, in a single sample taken from a test subject. Measurements for different lysophospholipids taken either simultaneously or sequentially from a single sample can improve the sensitivity and/or specificity of detection of cancer using the methods of the invention, reducing the occurrence of false negative or positive results.

The invention is based, at least in part, on the discovery that lysophospholipids, such as LysoPC and LysoPA, are increased in the bodily fluids of subjects having gynecological cancer, for example ovarian cancer.

The lysophospholipids for use in the methods of the invention have a glycerol backbone with a phosphate or a derivatized phosphate such as choline, inositol, ethanolamine, glycerol, or serine at the sn-3 position and a single tatty acid chain located at the sn-1 or sn-2 position, and linked to the glycerol backbone by an acyl linkage, with a hydroxyl at the other sn-1 or sn-2 position. Alternatively, a long chain alcohol is linked to the glycerol backbone at the sn-1 position by an alkyl or alkenyl linkage with a hydroxyl at the sn-2 position.

These compounds have the following general structures:

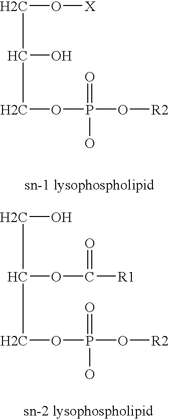

where X is any fatty acid or long chain alcohol including, but not limited to 18:0, 16:0, 18:1, 18:2, 20:4n-6 and 22:6n-3, attached through an acyl, alkyl or alkenyl bond; and where R1 is any fatty acid including, but not limited to, palmitic, palmitoleic, stearic, oleic, linoleic, arachidonic, and docasahexanoic fatty acid linked to the glycerol backbone of the phospholipid via an acyl bond.

R2 can be any derivative phosphate including, but not limited to, hydrogen, choline, inositol, ethanolamine, glycerol and serine.

Lysophospholipids for detection using the methods of the invention include, but are not limited to, LysoPA, LysoPC, LysoPS, LysoPE, LysoPI and LysoPG.

In another embodiment of the invention for prognosis of cancer in a subject, concentrations of lysophospholipids are measured over successive time intervals in subjects having cancer, and the concentrations of these compounds are compared over time to determine the prognosis of the cancer as well as the success of therapy. An increase in the concentration of lysophospholipid in a sample taken from the test subject at a later time indicates an increase in the number of viable tumor cells and a decrease in the concentration of lysophospholipid indicates a decrease in the number of viable tumor cells.

In yet another embodiment of the methods of the invention, the concentration of at least one other type of lysophospholipid is measured either simultaneously with the first type of lysophospholipid in the sample from the subject, or sequentially, to improve the sensitivity and/or specificity of detection of cancer in the subject.

In still another embodiment of the invention, the concentration of additional cancer cell markers such as CA125, are determined to further improve the sensitivity and/or specificity of the detection of cancer.

In a particular embodiment of the invention, concentrations of LysoPC and/or LysoPA, are measured in a sample of plasma taken from a test subject to diagnose the presence of a gynecological tumor in the subject. Diagnosis may also be performed by determining the rate of change over time of the concentration of a lysophospholipid in the sample from the subject.

Yet another embodiment is a diagnostic kit containing reagents for measuring concentrations of lysophospholipids and optionally including anti-lysophospholipid antibodies.

An advantage of the present invention is that it enables detection of cancers associated with the presence of certain lysophospholipids at an early stage and increases the specificity and sensitivity of detection, thus facilitating early intervention for an improved prognosis for the subject.

### 4. BRIEF DESCRIPTION OF THE DRAWINGS

FIGS. 1A-1C. Graphs depicting concentrations of phosphatidylcholine (PC) (1A) and LysoPC (1B) and ratio of LysoPC/PC (1C) in the plasma of control subjects and cancer patients. Data are shown as mean±SE for 7 controls and 17 ovarian cancer patients. Significant differences were observed for the LysoPC concentration (**p<0.01) and LysoPC/PC molar ratio (**p<0.01) for plasma from ovarian cancer patients as compared with controls.

FIG. 2. Graph depicting fatty acid compositions (mol % of total fatty acids) of plasma PC in control subjects and ovarian cancer patients. (16:0=palmitic acid; 18:0=stearic acid; 18:1=oleic acid; 18:2n-6=linoleic acid; 20:4n-6=arachidonic acid).

FIG. 3. Graph depicting fatty acid compositions (mol % of total fatty acids) of plasma LysoPC in control subjects and ovarian cancer subjects. Significantly higher concentrations were observed for palmitic (16:0) (**p<0.01) and stearic (18:0) acids (***p<0.001), and lower concentrations for oleic (18:1) (*p<0.05) and linoleic (18:2n-6) acids (***p<0.001) in ovarian cancer subjects as compared to controls.

FIG. 4. Graph depicting molar ratios of 16:0 (palmitic acid/18:2n-6 (linoleic acid) in plasma PC and LysoPC of control subjects and ovarian cancer subjects. Data are shown as means±SE for 7 controls and 17 ovarian cancer patients. Significant differences were observed in the case of plasma LysoPC (***p<0.001) as compared to plasma PC.

FIG. 5. Graph showing values for [LysoPC/PC]×[palmitoyl-LysoPC(16:0/linoleoyl-LysoPC(18:2n-6)] in ovarian cancer patients and controls (“normals”). Vertical lines show mean±SE for 7 controls and 17 ovarian cancer patients. Significantly higher values were observed in plasma from ovarian cancer patients as compared to controls (***p<0.001).

FIG. 6. Graph showing concentrations of LysoPA in plasma from ovarian cancer patients and control subjects. Vertical lines show mean±SE for 9 controls and 52 ovarian cancer patents. Significantly higher concentrations of LysoPA were observed in plasma from ovarian cancer patients as compared to controls. (***p<0.001).

FIG. 7. Graph depicting concentrations of LysoPA in the plasma of patients with active disease and quiescent disease as compared to controls. The left 3 bars represent total LysoPA and the right 3 bars represent LysoPA with polyunsaturated fatty acids only. Bars show mean±SE for 9 controls and 52 ovarian cancer patients. Significantly higher concentrations of LysoPA and LysoPA with saturated fatty acids were found in patients with active disease as compared to patients in the quiescent stage of the disease or as compared to controls.

### 5. DETAILED DESCRIPTION OF THE INVENTION

The present invention provides methods for screening for cancers correlated with elevated concentrations of lysophospholipids, including, but not limited to, lysophosphatidic acid (LysoPA), lysophosphatidyl choline (LysoPC), lysophophatidyl serine (LysoPS), lysophosphatidyl inositol (LysoPI), lysophosphatidyl ethanolamine (LysoPE) and lysophosphatidyl glycerol (LysoPG) in a sample of bodily fluid from a subject. The subject may be a non-human, or preferably, a human animal.

The cancers correlated with increased concentrations of these lysophospholipids include, but are not limited to, gynecological tumors including tumors of the ovaries, cervix, and uterus. Certain cancers such as leukemia are not correlated with increased concentrations of these lysophospholipids. Thus, the methods of the present invention are directed to the detection of cancers that are known to correlate or shown to correlate with increased concentrations of lysophospholipids in the bodily fluids from a subject.

The compounds useful in the methods of the invention are lysophospholipids having a glycerol backbone with a phosphate or a derivatized phosphate such as choline, inositol, ethanolamine, glycerol, or serine at the sn-3 position and a single fatty acid chain located at the sn-1 or sn-2 position, and linked to the glycerol backbone by an acyl linkage, with a hydroxyl at the other sn-1 or sn-2 position. Alternatively, a long chain alcohol is linked to the glycerol backbone at the sn-1 position by an alkyl or alkenyl linkage with a hydroxyl at the sn-2 position.

These compounds have the following general structures:

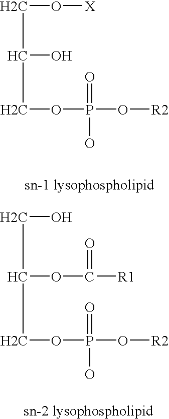

where X is any fatty acid or long chain alcohol including, but not limited to 18:0, 16:0, 18:1, 18:2, 20:4n-6 and 22:6n-3, attached through an acyl, alkyl or alkenyl bond; and where R1 is any fatty acid including, but not limited to, palmitic, palmitoleic, stearic, oleic, linoleic, arachidonic and docasahexanoic fatty acid linked to the glycerol backbone of the phospholipid via an acyl bond.

R2 can be any derivative phosphate including, but not limited to, hydrogen, choline, inositol, ethanolamine, glycerol and serine.

These lysophospholipids share the property of having growth-promoting or signaling activity toward cancer cells in vitro and in vivo. For example, this activity is associated with increases in cytosolic free calcium (Xu et al., Clin. Cancer. Res. 1:1223-1232 (1995)), or activation of other signaling pathways (Moolenaar, Current Opinion in Cell Biol. 7:(2):203-210 (1995); Moolenaar, J. Biol. Chem. 270(22):12949-12952 (1995); Jalink et al., Biochim. Biophys. Acta 1198(2-3):186-196 (1994); and Xu et al., Biochem. J. 309:933-940 (1995)).

### 5.1 Uses of the Invention

The methods of the invention can provide a number of benefits. First, the methods provide a rapid and economical screen for large numbers of subjects to promote early diagnosis of cancer which can result in improved quality of life and better survival rates for cancer patients.

Using the methods of the invention for prognosis, the medical professional can determine whether a subject with cancer in the early stages requires therapy or does not require therapy. This could also identify subjects who may not benefit from a particular form of therapy e.g. surgery, chemotherapy, radiation or biological therapies. Such information could result in improved therapy design for obtaining better responses to therapy, improved quality of life and improved survival for the cancer patient.

The methods of the invention can also be used to identify patients for whom therapy should be altered from one therapeutic agent to another. This could obviate the need for “second look” invasive procedures to determine the patient's response to the therapy and facilitate decisions as to whether the particular type of therapy should be continued, terminated or altered.

Because cancers will recur in a significant number of patients with advanced cancers, early detection and continued monitoring over time using the methods of the invention, could identify early occult (i.e. “hidden”) recurrences prior to symptoms presenting themselves. Use of the methods of the invention for these purposes can also result in improved responses to therapy, improved quality of life and improved survival for cancer patients.

In addition, the methods of the invention will facilitate distinguishing benign from malignant tumors. Masses in an organ such as the ovary can be initially detected using procedures such as ultrasound or by physical examination. Thereafter, the methods of the invention can be used to diagnose the presence of cancer. This could obviate the need for surgical intervention, and/or identify subjects where continued monitoring is appropriate resulting in improved early detection and survival for cancer patients.

Yet another use for the methods of the invention is to determine the origin of an unknown primary tumor. The tissue of origin of malignant tumors in the peritoneal cavity and in other parts of the body frequently cannot be determined using conventional techniques. This information is useful to direct the medical professional to the most appropriate therapy for the tumor. Measuring concentrations of lysophospholipids and/or certain types of lysophospholipids using the methods of the invention could provide information about the tissue of origin for a tumor. For example, elevated concentrations of lysophospholipids could distinguish between lymphomas and bowel tumors which may have lower concentrations of lysophospholipids than gynecological tumor.

In addition to determination of the concentrations of lysophospholipids associated with cancers, using the methods of the invention, measurement may be made of other cancer cell markers including, but not limited to CA125, Tac, soluble IL2 receptor alpha, mCSF, OVX1, CEA, PSA, CA15-3, CA19.9, to improve the sensitivity and/or the specificity of detection of cancer.

Particularly useful measurements for increasing sensitivity are measurements of the concentrations of lysophospholipid and other cancer markers taken over time or in units of rate of change of the lysophospholipid over time to decrease false positive results.

Moreover, the information on concentrations of lysophospholipids determined by the methods of the present invention, may suggest additional procedures be instituted such as use of ultrasound, biopsy, laparascopy or surgery, to improve the detection of early cancer and to screen large populations of subjects for the presence of cancer.

### 5.2. Methods of Detecting Lysophospholipids and/or their Constituent Fatty Acids to Diagnose Cancer

The invention provides for methods to diagnose the presence of cancer in a subject. In a particular embodiment, the invention provides a method for detecting increased concentrations of lysophosphatidylcholine (LysoPC) and lysophophatidic acid (LysoPA) in a sample of bodily fluid taken from a test subject. The bodily fluid may be plasma, serum, urine, saliva, ascites, cerebral spinal fluid or pleural fluid.

The methods of the invention are carried out as follows. The concentration of lysophospholipid as defined above is measured after lipid extraction and analysis, or by an antibody based assay, as described further infra. The measurements may be taken as 1) the concentration of the specific lysophospholipid selected, e.g. LysoPC or LysoPA present in the sample from the subject; 2) the concentration of a subtype of the selected lysophospholipid having a particular degree of saturated or unsaturated fatty acids and/or fatty acid chain length (e.g. palmitoyl-LysoPC or linoleoyl-LysoPC) or the concentration of a subtype having a particular long chain alcohol attached to the glycerol backbone; 3) the concentration of total lysophospholipids present in a sample; or 4) the concentration of first one lysophospholipid, e.g., LysoPC, followed by measurement of another lysophospholipid, e.g. LysoPA, in a single sample taken from a test subject. Measurements for different lysophospholipids taken either simultaneously or sequentially from a single sample can improve the sensitivity and/or specificity of detection of cancer using the methods of the invention, reducing the occurrence of false negative or positive results.

The measurement of lysophospholipids can be determined as a concentration (i.e. the amount of lysophospholipid present relative to liquid volume of the sample (e.g. μmol/ml) or is used after normalization to the concentration of other compounds in the subject's sample including other lysophospholipids, phospholipids, albumen and creatinine.

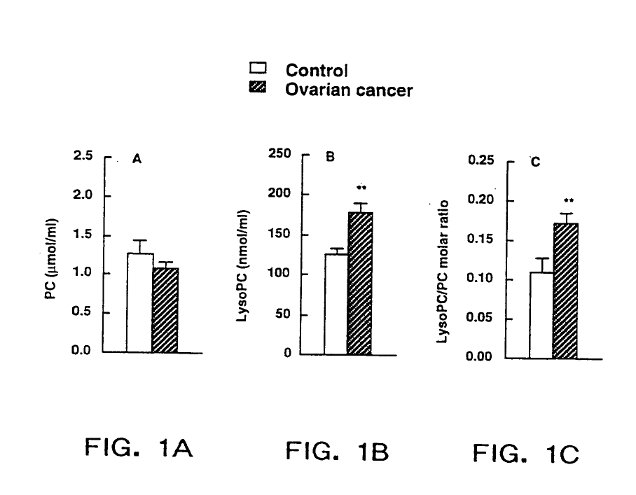

For example, concentrations of a lysophospholipid such as LysoPC having specific types of saturated fatty acid chains such as stearic or palmitic are measured and compared to concentrations of the lysophospholipid having different types of fatty acid chains, e.g. LysoPC having unsaturated fatty acid chains such as oleic and linoleic. These values are then normalized using the total amount of a component such as phosphatidylcholine (PC) in the sample. Such measurements may provide more specific or sensitive indications of the presence of cancer than measurements of the total lysophospholipid, e.g. LysoPC, alone, without regard to fatty acid chain types. Moreover, concentrations of a lysophospholipid could be compared to the equivalent phospholipid, e.g. concentrations of LysoPC could be compared to concentrations of PC in the sample (see Examples, infra and FIGS. 1-5).

Initially, physiological (“normal”) concentrations of lysophospholipids and/or specific lysophospholipid species are determined in subjects not having cancer. Subsequently, the concentration of the lysophospholipids are measured in a sample of bodily fluid from a test subject to be screened for cancer and compared to the concentrations established for normal subjects. Where the concentrations of lysophospholipids are elevated relative to normals, a diagnosis of the presence of cancer may be made. Additionally, as detailed above, the concentrations may be compared after normalization to the concentration of other compounds.

The concentration of a lysophospholipid detected in the sample taken from a subject may be measured by first extracting lipids as described in detail infra. The amount of lysophospholipid is then quantified using standard procedures such as gas chromatography HPLC, ELIZA, NMR or other approaches. Alternatively, the presence of lysophospholipids in a sample is quantified using an anti-lysophospholipid antibody in an antibody based assay, as also described infra. Concentrations of lysophospholipid that are significantly increased relative to normal controls, for example one or more standard deviations above normal, may indicate the presence of cancer.

As an additional diagnostic tool, the concentrations of selected lysophospholipid species are measured and normalized as described above. Increased concentrations for these species may indicate the presence of cancer. This may increase the sensitivity and specificity of the assay.

The concentrations of lysophospholipids determined using the methods of the invention, can be used to diagnose and screen subjects for the presence of cancer, as well as to determine the prognosis of a subject with cancer. Moreover, the response of cancer to treatment may be monitored by determining concentrations of lysophospholipid in samples taken from a subject over time.

Additionally, the rate of change in concentrations of lysophospholipids over time can also be determined, and may provide a more sensitive or specific indication of the presence of cancer.

A variety of methods can be employed for the diagnostic and prognostic evaluation of cancer. For example in vitro diagnostic assay methods of the invention include detection of the phospholipid in a biological sample, and may, therefore, be used as part of a diagnostic or prognostic technique whereby patients are tested for abnormal concentrations of lysophospholipid. Such assay methods include well-known techniques in the art such as gas chromatography, NMR and HPLC. For example, lipids may be extracted from the test sample of bodily fluid using extraction procedures such as those described by Bligh and Dyer, Can. J. Biochem. Physiol. 37:911-917 (1959), incorporated by reference herein. Thin-layer chromatography may be used to separate various phospholipids, for example as described by Thomas and Holub, Biochim. Biophys. Acta, 1081:92-98 (1991), incorporated by reference herein.

Phospholipids and lysophospholipids are then visualized on plates, for example using ultraviolet light as described by Gaudette et al., J. Biol. Chem. 268:13773-13776 (1993), incorporated by reference herein. Fatty acids are detected by extraction from the visualized phospholipids and may be quantified using a procedure such as gas chromatography (see Skeaff and Holub, In M. Lagarde (ed.), Biology of Eicosanoids, Vol. 152, pp. 63-76, Inserm, Paris (1987), incorporated by reference herein, HPLC or NMR. The concentrations of the lysophospholipids comprised of the fatty acids can be derived from the fatty acid content assessed by gas chromatography and calibrated with an internal standard such as heptadecanoic acid. Alternatively, lysophospholipid concentrations can be identified by NMR or HPLC following isolation from phospholipids or as part of the phospholipid.

In addition to direct measurement of concentrations of lysophospholipids by extraction, antibodies, such as monoclonal antibodies reactive with lysophospholipids, can be used in an assay to detect concentrations of lysophospholipids in test sample. For example, anti-phospholipid antibodies may be labeled using standard procedures and used in assays including radioimmunoassays (RIA), both solid and liquid phase, fluorescence-linked assays or enzyme-linked immunosorbent assays (ELISA) wherein the antibody is used to detect the presence of the lysophospholipid in the fluid sample (see, e.g. Uotila. et al., J. Immunol Methods 42:11 (1981)), and fluorescence techniques (Sikora et al., (eds)., Monoclonal Antibodies, pp. 32-52, Blackwell Scientific Publications, (1984)).

Monoclonal antibodies raised against lysophospholipids for use in assays to detect lysophospholipids may be produced according to established procedures, e.g. by immunization of various host animals with the lysophospholipid, fragments thereof or functional equivalents thereof. Such host animals include, but are not limited to, rabbits, mice, rats, goats, to name but a few. Various adjuvants may be used to increase the immunological response in the host animal, depending on the host species, including, but not limited to, Freund's (complete and incomplete), mineral gels such as aluminum hydroxide, surface active substances such as pluronic polyols, polyanions, peptides, oil emulsions, keyhole limpit hemocyanin, dinitrophenol and potentially useful human adjuvants such as BCG (Bacille Calmette-Guerin) and Corynebacterium oarvum. Polyclonal antibodies are heterogeneous populations of antibody molecules derived from the sera of the immunized animals.

Monoclonal antibodies, which are homogeneous populations of antibodies to a particular antigen, may be obtained by any technique which provides for the production of antibody molecules by continuous cell lines in culture or use of phage display libraries. These include, but are not limited to, the hybridoma technique of Kohler and Milstein (Nature 356:495-497 (1975)), the human B-cell hybridoma technique (Kosbor et al., Immunology Today 4:72 (1983); Cole et al., Proc. Nat'l. Acad. Sci. USA 80:2026-2030 (1983)), and the EBV-hybridoma technique (Cole et al., Monoclonal Antibodies And Cancer Therapy, Alan R. Liss, Inc., pp. 77-96 (1985)). Such antibodies may be of any immunoglobulin class including IgG, IgM, IgE, IgA, IgD and any subclass thereof. The hybridoma producing the mAb may be cultivated in vitro or in vivo.

Antibody fragments which recognize specific lysophospholipids may be used and are generated by known techniques. For example, such fragments include, but are not limited to: the F(ab′)₂ fragments that can be produced by pepsin digestion of the antibody molecule and the Fab fragments that can be generated by reducing disulfide bridges of the F(ab′)₂ fragments. Alternatively, Fab expression libraries may be constructed (Science 246:1275-1281 (1989)) to permit rapid and easy identification of monoclonal Fab fragments having the desired specificity.

Procedures for preparing antibodies against lysophospholipids for use in the above-described assays have been described, for example for producing phosphatidylinositol and phosphatidic acid antibodies (See, Keating et al., Biochem. J. 317(Pt. 3):643-646 (1996); Fukami et al., Proc. Nat'l. Acad. sci. USA 85:9057-9061 (1988); Fukami et al., Proc. Nat'l. Acad. Sci. USA 85(23):9057-9061 (1988); and Matuoka et al., Science, 239(4840):640-643 (1988)).

### 5.3 Methods for Monitoring Subjects having Cancer

The invention also provides methods for following cancer in a patient over time. For example, the concentration of a lysophopholipid such as LysoPC in a sample of bodily fluid from a cancer patient is determined. At a later time, the concentration of that lysophospholipid is measured and compared to the concentration taken at the earlier time from that patient. If there is an increase in the concentration of lysophospholipid over time, it may indicate an increase in the number of viable tumor cells, and thus an increase in the cancer present in the patient. Conversely, if there is a decrease in the concentration of lysophospholipid, it may indicate a decrease in the cancer presence. Additionally, measurement of more than one type of lysophospholipid, eg LysoPA, may be taken from each sample. These measurements can provide information for the medical professional to adjust therapy to alter, discontinue or commence certain therapeutic agents or procedures to improve prognosis and survival for the patient.

### 5.4 Diagnostic Kits

The methods described herein for measuring concentrations of lysophospholipids in samples of bodily fluids from a subject may also be performed, for example, by using pre-packaged diagnostic kits. Such kits include reagents for assessing the concentration of lysophospholipid, for example, reagents for extracting lipids from various liquid samples. The reagents include ancillary agents such as buffering agents, and agents such as EDTA to inhibit subsequent production or hydrolysis of lysophospholipids during transport or storage of the samples. Alternatively, the diagnostic kit can include labeled antibody reagents such as anti-lysophospholipid antibodies, or combinations of antibodies, that may be conveniently used, e.g. in a clinical setting, to diagnose subjects with cancer. The kits may also include an apparatus or container for conducting the methods of the invention and/or transferring samples to a diagnostic laboratory for processing, as well as suitable instructions for carrying out the methods of the invention.

The following examples are presented to demonstrate the methods of the present invention and to assist one of ordinary skill in using the same. The examples are not intended in any way to otherwise limit the scope of the disclosure or the protection granted by Letters Patent granted hereon.

### 6. EXAMPLE

### Detection of LysoPC and its Constituent Fatty Acids in the Plasma of Ovarian Cancer Patients

### 6.1 Subjects

Seventeen subjects diagnosed with epithelial ovarian cancer aged 36 to 74 years (mean 55 years), who were admitted to the Toronto General Hospital were included in this example. Seven of these subjects were diagnosed with active disease. Nine subjects were receiving cisplatinum based chemotherapy at the time of analysis. Six subjects with active leukemia whom were between cycles of chemotherapy at M.D. Anderson Cancer Center (Houston, Tex.) were also included for comparison. Control subjects were seven healthy women aged 46 to 63 years (mean 52 years).

### 6.2 Plasma Samples

Blood (7 ml) was drawn into vacutainers (Becton Dickinson and Company, Rutherford, N.J.) containing EDTA and plasma was separated (1,600×g, 15 min). The plasma was stored at −20° C. and analyzed within 7 days.

### 6.2 Lipid Extraction and Phospholipid Analysis

Plasma lipids were extracted by the method of Bligh and Dyer, Can. J. Biochem . Physiol. 37:911-917 (1959), incorporated by reference herein, with some modifications. To each 0.5 ml plasma sample, 3.75 ml chloroform/methanol (1:2 v/v) was added and the contents vortexed for 1 min. After centrifugation to pellet the majority of the plasma proteins, the chloroform/methanol extracts were transferred to new tubes and mixed thoroughly with 1.25 ml chloroform. 1.75 ml H₂O was added, the contents vortexed briefly, and phase separation was accomplished by centrifugation. After removing the lower chloroform layer (the first extracts), 2.5 ml chloroform and 63 μl concHCl were added to the remaining aqueous phases. The chloroform layer was collected after centrifugation (the 2nd extracts). The first and the second (acidified) chloroform extracts were concentrated under nitrogen and spotted on silica gel plates (Silica Gel 60) (EM Science, Gibbstown, N.J.). The phospholipids were separated by two-dimensional TLC [dimension 1: chloroform/methanol/14.8N ammonium hydroxide:65:35:5.5 (v/v/v) and dimension 2: chloroform/methanol/88% formic acid/water: 55:28:5:1 (v/v/v/v)] according to the method of Thomas and Holub, Biochim. Biophys. Acta, 1081:92-98 (1991), incorporated by reference herein. TLC plates were dried at 40° C. for 30 minutes under nitrogen between the two chromatographic steps. Phospholipids were detected by spraying the plates with 0.1% 8-anilino-1-naphthalene-sulfonic acid (ANS) (Sigma, St. Louis, Mo.) in water and viewing under ultraviolet light (Gaudette et al., J. Biol. Chem., 268:13773-13776 (1993)). The spots corresponding to PC and LysoPC were scraped from the plates and transmethylated in the presence of silica gel for 2 hours at 85° C. using 2 ml of acetyl chloride/methanol 5:50 (v/v). A known amount of heptadecanoic acid (17:0) was used as an internal standard. Following transmethylation, the fatty acid methyl esters were extracted with petroleum ether and quantified using a model 5890 A gas chromatograph (Hewlett Packard, Wilmington, Del.), as previously described (Skeaff and Holub, In M. Lagarde, (ed), Biology of Eicosanoids, Vol. 152, pp. 63-76, Inserm, Paris (1987)), incorporated by reference herein. Concentrations of PC and LysoPC presented herein were derived from the fatty acid content assessed by gas chromatography (GC) and calibrated with heptadecanoic acid.

Data were analyzed by Student's t-test and significant differences indicated when p<0.05.

### 6.3 Results

### 6.3.1 Plasma PC and LysoPC Concentrations

Although plasma PC concentrations were, on average, 14% lower in ovarian cancer subjects (1.08±0.07 μmol/ml, mean±SE) than in normal controls (1.26±0.17 μmol/ml), this difference was not significant (p<0.2) (FIG. 1A). In contrast, plasma LysoPC concentrations in ovarian cancer subjects were significantly higher (on average 43%; p<0.01) than in controls (FIG. 1B). The corresponding values were 125.6±7.1 nmol/ml (mean±SE) and 179.7±10.0 nmol/ml for the controls and ovarian cancer subjects. The molar ratio of LysoPC to PC was also markedly higher in ovarian cancer patients (0.17±0.01, mean±SE) in comparison to control subjects (0.11±0.02) (FIG. 1C).

Of the total plasma PC and LysoPC obtained by this procedure, more than 98% and 88%, respectively, were extracted into the first chloroform (neutral extracts) in both control and ovarian cancer subjects. Although contributing only a minor amount to the total plasma PC, a significantly greater amount (21.6 vs 14.4 nmol/ml; p<0.05) was observed in the acidified extract of plasma from ovarian cancer subjects than from normal controls.

### 6.3.2 Fatty Acid Composition of Plasma PC and LysoPC

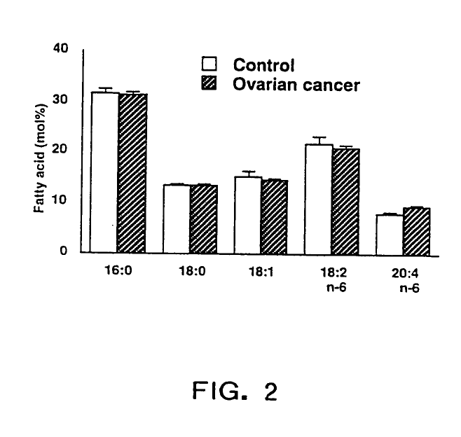

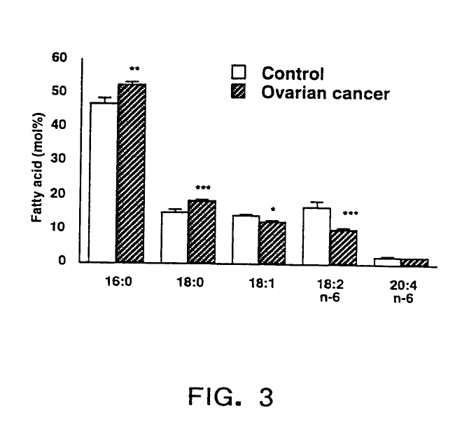

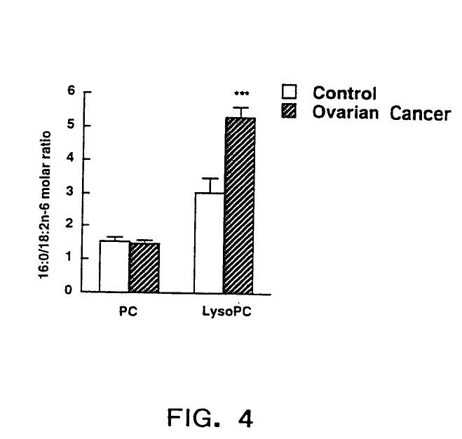

There were no significant differences in the fatty acid compositions of plasma PC between controls and ovarian cancer patients (FIG. 2). In contrast, plasma LysoPC from ovarian cancer subjects contained significantly higher concentrations of palmitoyl- and stearoyl-LysoPC species and lower concentrations of oleoyl- and particularly linoleoyl-LysoPC species than controls in terms of mol% of total fatty acids. (FIG. 3). The molar ratio of plasma palmitoyl- to linoleoyl-LysoPC in ovarian cancer subjects (5.3±0.3, mean±SE) was significantly higher than those of controls (3.0±0.4) (FIG. 4).

### 6.3.3 Potential of Comparison of Concentrations of Plasma Palmitoyl-LysoPC to Linoleoyl-LYsoPC Molar Ratios as an Ovarian Cancer Indicator

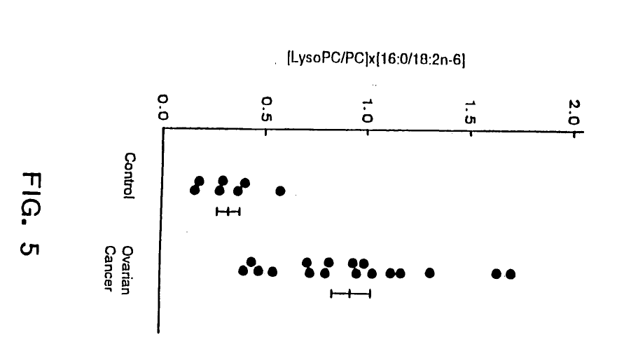

Although plasma LysoPC concentrations (FIG. 1B) and molar ratios of LysoPC (FIG. 1C) and palmitoyl-LysoPC/linoleoyl-LysoPC (FIG. 4) showed significantly higher concentrations in ovarian cancer subjects as compared to normal controls; p<0.01, p<0.01 and p<0.001, respectively, the data obtained from some subjects overlapped with those from controls. Therefore, values of [LysoPC/PC molar ratio]×[palmitoyl-LysoPC/linoleoyl-LysoPC molar ratio] were calculated. The values between controls (0.324±0.054, mean±SE) were compared with ovarian cancer subjects (0.928±0.092, mean±SE). As shown in FIG. 5, the average values in subjects with ovarian cancer were markedly higher (p<0.001) than in controls. Furthermore, 15 of 17 subjects had higher values than the mean±1SD (0.0450) of the controls and 13 of 17 subjects had greater than the mean±2SD (0.596) of controls.

### 7. EXAMPLE

### Detection of LysoPA and Its Constituent Fatty Acids in the Plasma of Ovarian Cancer Patients

### 7.1 Subjects and Plasma Samples

Blood was collected from 52 consecutive ovarian cancer patients at the Gynecological Oncology Clinic of the Toronto Hospital. Blood was collected in EDTA containing tubes to decrease metabolism or production of lysophospholipids or phospholipids. Normal samples were obtained from nine (9) healthy volunteers as controls. Samples were centrifuged as described above to remove platelets and other blood components and plasma frozen at −20° C. Plasma was assayed for LysoPA and other lysophospholipids as described below. Patients were assessed for presence of active or quiescent disease based on clinical findings.

### 7.2 LysoPA Purification

LysoPA was purified as described above for LysoPC, except that only the acidified chloroform extracts or second acidified chloroform extracts were assessed.

### 7.3 LysoPA Fatty Acid Analysis

LysoPA, resolved by TLC was transmethylated in the presence of silica gel for 2.5 hour at 85° C. using 2 ml of acetyl chloride/methanol 5:50 (v/v). Heptadecanoic acid (17:0) was used as an internal standard. Following 20 transmethylation, the fatty acid methyl esters, derived from fatty acids contained in LysoPA, were extracted with petroleum ether and quantified by gas chromatography (GC) on a model 5890A gas chromatograph (Hewlett Packard, Wilmington, Del.), as described by Skeaff and Holub (Skeaff and Holub, In: M. Lagarde (ed.) Biology of Eicosanoids and Related Substances in Blood and Vascular Cells, 152:63-76, Paris, Inserm (1987) incorporated by reference herein. All concentrations of LysoPA presented herein are derived from fatty acid content assessed by GC and calibrated with heptadecanoic acid.

### 7.4 Results

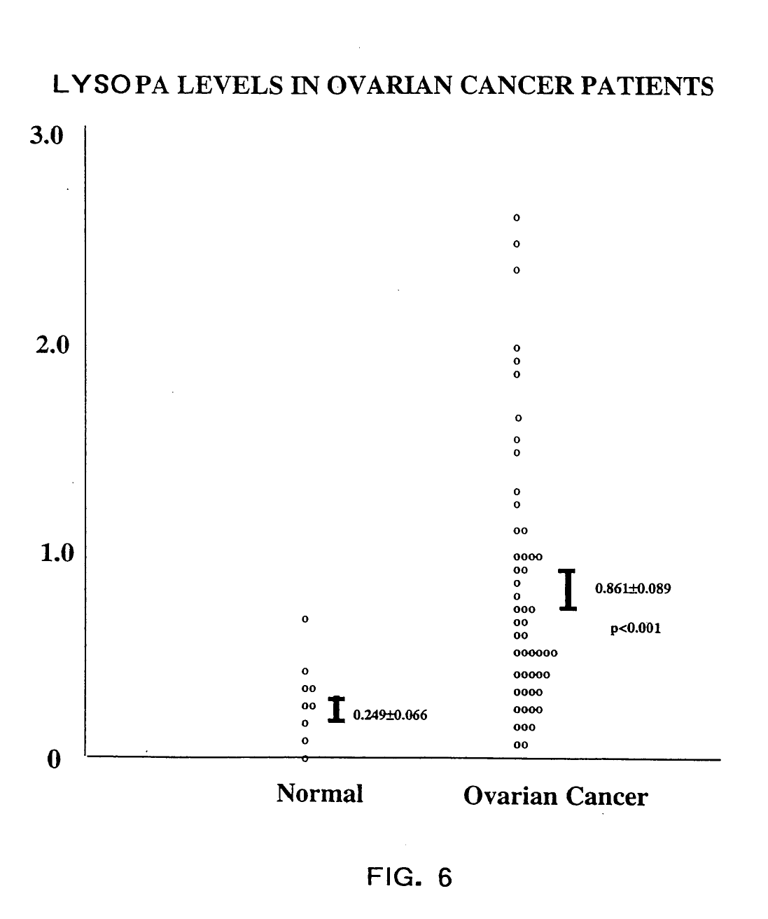

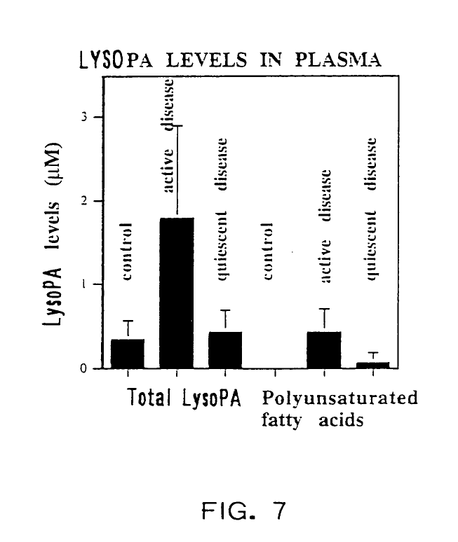

This example demonstrates that concentrations of total LysoPA were markedly elevated in ovarian cancer patients as compared to normal subjects (as shown in FIG. 6). Moreover, when plasma samples from patients shown in FIG. 6 were assessed for the presence of active or quiescent disease, that amount of total LysoPA and LysoPA with polyunsaturated fatty acids were significantly higher than the equivalent concentrations of these compounds in the plasma from normal subjects. Concentrations of LysoPA with polyunsaturated fatty acid chains are increased in some patients with quiescent disease as indicated in FIG. 7. Because ovarian cancer frequently recurs, these results may reflect patients with occult tumor present which could not be detected by clinical analysis.

### 7.5 Discussion

Lysophospholipids (LPAs) such as LysoPC and LysoPA are a normal constituent of human plasma. Much of the LysoPC in plasma is bound to albumin (Skipski et al., Biochem. J. 104(2):340-52 (1967)). The absolute concentration of LysoPC in the plasma of healthy individuals varies considerably between studies (Skipski et al., supra; Philips and Dodge, J. Lipid Res. 8:676-681 (1967); Gillet and Besterman, Atherosclerosis 22:111-124 (1975); Kriat et al., J. Lipid REs. 34:1009-1019 (1993)). This variability appears to reflect differences in total plasma lipid content between study populations, because when expressed as a percentage of total plasma phospholipid, LysoPC is consistently observed to be present at approximately 6.5%. The estimated value for plasma LysoPC in healthy controls (assuming PC represents 68% of total plasma phospholipid; Skipski et al., supra; Gillett and Besterman, supra) is 7.5±1.0% (mean±S.E.) of total phospholipid, which is in general agreement with previous literature values. The moderately higher value in the present study may reflect the use of a second acidified extraction step in which approximately 12% of the total LysoPC was recovered. In contrast, LysoPC is present at 11.8±0.8% of total phospholipids in the plasma of patients with ovarian cancer. And, LysoPA is present at 0.05% of total phospholipids in the plasma of patients with ovarian cancer. On a relative basis, for LysoPC, palmitoyl and stearoyl-LysoPC species are increased, oleoyl and linoleoyl-LysoPC species decreased, and aracidonoyl-LysoPC unchanged in the plasma of ovarian cancer patients as compared to healthy controls.

It is apparent from the results of these examples that concentrations of LysoPA, in particular LysoPA with polyunsaturated fatty acids, are elevated in plasma obtained from ovarian cancer patients as compared to plasma from normal controls. The elevations in concentrations of LysoPA, particularly LysoPA with polyunsaturated fatty acids, were more marked in individuals with active disease, either defined by the presence of ascites, radiographic evidence of disease or by physical exam. This increase is obvious from a scatter plot (FIG. 6) showing that concentrations of LysoPA are elevated in a significant proportion of ovarian cancer patients. This increase is more apparent when patients are segregated into those with currently active disease and those with currently inactive disease (FIG. 7). A significant proportion (approximately 50%) of those subjects with inactive or quiescent disease will recur within two years. This may be indicated by the increased concentrations of LysoPA and in particular concentrations of LysoPA with polyunsaturated fatty acids indicated in FIG. 7. Thus, measuring concentrations of LysoPA, particularly concentrations of LysoPA with polyunsaturated fatty acids in plasma may provide a method for indicating response to therapy as well as in the early detection of recurrence. Given that measuring concentrations of CA125 in patients undergoing therapy or with quiescent disease can be extrapolated to studies of patients prior to diagnosis, these results indicate that measuring concentrations of LysoPA, particularly concentrations of LysoPA with polyunsaturated fatty acids, will provide sufficient sensitivity and specificity to be used in the methods of the invention for early screening of subjects for the presence of ovarian cancer. Measurement of concentrations of LysoPA, particularly concentrations of LysoPA with polyunsaturated fatty acids, may either be used alone or in combination with studies of multiple markers, including but not limited to LysoPC, other lysophospholipids, CA125, mCSF, TAC, soluble IL2 receptor alpha and other known and unknown markers, to provide high sensitivity and/or specificity for the detection of early ovarian cancer.

While not wishing to be bound by any particular theory, it is likely that increased LysoPC and LysoPA production may be responsible for the elevated plasma concentrations of these lipids. While the source(s) and mechanism(s) responsible for the elevation of LysoPC and LysoPA in the plasma of ovarian cancer patients are not known, Applicants believe that the ovarian cancer cells may be the source of the increased lysophospholipids. Increased phospholipase A₁ (PLA₁) or PLA₂ or PLD activity would be compatible with the elevated plasma concentrations of LysoPC and of LysoPA observed in the present examples. Because PLA₂ cleaves fatty acids from the sn-2 position of PC resulting in LysoPC containing primarily saturated fatty acids, it may account for the increase in saturated species of LysoPC (palmitoyl and stearoyl). Since PLA₁ cleaves fatty acids from the sn-1 position, it may account for the LysoPA with primarily unsaturated fatty acids (lineoyl, arachidonic, DHA). This implies a role for phospholipases, and, as the increased LysoPC contains primarily saturated fatty acids, a role for phospholipase A₂ (PLA₂), and as the increased LysoPA contains unsaturated fatty acids, a role for PLA₁. PLD could play a role in converting LysoPC, LysoPS, LysoPI, LysoPE and LysoPG to LysoPA.

LysoPC and LysoPA have been proposed to activate cells from a number of lineages. This example demonstrates that LysoPC and LysoPA concentrations are significantly elevated relative to normal controls in the plasma of ovarian cancer patients. This phenomenon does not appear to be common to all cancers as five out of six leukemia patients tested had markedly lower (less than one half of normal) levels of plasma LysoPC than those in samples from normal controls. In the plasma of ovarian cancer patients, the percentage of palmitoyl- and stearoyl-LysoPC species are significantly higher, whereas oleoyl and particularly linoleoyl-LysoPC are significantly lower than in control subjects. The molar ratios of LysoPC/PC and palmitoyl-LysoPC/linoleoyl-LysoPC are also significantly elevated in the plasma of ovarian cancer patients as compared to those of control subjects. Furthermore, the calculated value of plasma [LysoPC/PC]×[palmitoyl-LysoPC/linoleoyl-LysoPC] is markedly higher in patients as compared to controls. Finally, concentrations of LysoPA and LysoPA with polyunsaturated fatty acids were higher in the plasma of ovarian cancer patients. These values may serve as an indicator for early diagnosis, prognosis, and monitoring therapy of ovarian cancer patients.

Various publications are cited herein which are hereby incorporated by reference in their entirety. As will be apparent to those skilled in the art in which the invention is addressed, the present invention may be embodied in forms other than those specifically disclosed above without departing from the spirit or potential characteristics of the invention. Particular embodiments of the present invention described above are therefore to be considered in all respects as illustrative and not restrictive. The scope of the present invention is as set forth in the appended claims and equivalents thereof rather than being limited to the examples contained in the foregoing description.

We claim:

1. A method for detecting the presence of cancer in a test subject, comprising determining the concentration of a lysophospholipid in a sample of bodily fluid taken from said test subject and comparing the concentration of lysophospholipid to the concentration of the lysophospholipid in samples from normal subjects lacking cancer, whereby an increase in the concentration of lysophospholipid in the sample from said test subject relative to the concentration of the lysophospholipid in samples from normal subjects indicates the presence of cancer.

2. The method of claim 1 further comprising the step of determining the concentration of at least one other type of lysophospholipid in the samples.

3. The method of claim 1 further comprising the step of determining the concentration of subtypes of the lysophospholipid in the samples.

4. The method of claim 3 wherein said method further comprises the step of measuring the concentrations of palmitoyl-X, stearoyl-X, oleoyl-X and linoleoyl-X in the sample from the test subject, where X is selected from the group consisting of LysoPC, LysoPA, LysoPS, LysoPI, LysoPE and LysoPG lysophospholipids, and comparing said concentrations to control concentrations.

5. The method of claim 4 further comprising determining the molar ratio of palmitoyl-X to linoleoyl-X in the sample and comparing said ratio to control ratios.

6. The method of claim 4 further comprising determining the value of [X/PC]×[palmitoyl-X/linoleoyl-X] in the sample from the test subject and comparing said value to control values.

7. The method of claim 1 further comprising the step of determining the concentration of additional cancer cell markers in the sample from the test subject.

8. The method of claim 7 wherein said additional cancer cell markers are selected from the group consisting of CA125, Tac, soluble IL2 receptor alpha, mCSF, OVX1, CEA, PSA, CA15-3 and CA19.9.

9. The method of claim 1 wherein said cancer is a gynecological cancer selected from the group consisting of ovarian, fallopian tube, uterine, intraperitoneal carcinomatosis and cervical cancers.

10. The method of claim 1 wherein said step of determining the concentration of lysophospholipid comprises contacting said sample with an anti-lysophospholipid antibody to bind with lysophospholipid and detecting said bound antibody.

11. The method of claim 1 further comprising the step of determining the fatty acid composition of said lysophospholipid and the concentration of said fatty acids of the lysophospholipid in the samples.

12. The method of claim 1 wherein said lysophospholipid is a sn-1 or sn-2 lysophospholipid having a glycerol backbone with a phosphate or derivatized phosphate at the sn-3 position and having a single fatty acid chain located at the sn-1 or sn-2 position linked by an acyl linkage and having a hydroxyl located at the other sn-1 or sn-2 position.

13. The method of claim 1 wherein said lysophospholipid is a sn-1 lysophospholipid having a glycerol backbone with a phosphate or derivatized phosphate at the sn-3 position and having a long chain alcohol located at the sn-1 position linked by an alkyl or alkenyl linkage and having a hydroxyl located at the sn-2 position.

14. The method of claim 12 or 13 wherein said lysophospholipid has the general structure of: 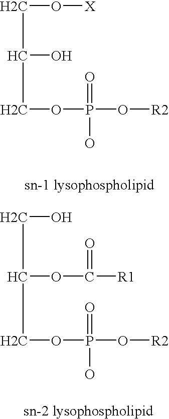
wherein X is a single chain fatty acid or long chain alcohol, and R1 is a single chain fatty acid and wherein
R2 is a derivatized phosphate.

15. The method of claim 14 wherein X is selected from the group consisting of 18:0, 16:0, 18:1, 18:2, 20:4n-6, 22:6n-3.

16. The method of claim 14 wherein R1 is selected from the group consisting of palmitic, palmitoleic, stearic, oleic, linoleic, arachidonic and docasahexanoic fatty acids.

17. The method of claim 14 where R2 is selected from the group consisting of hydrogen, choline, inositol, ethanolamine, glycerol and serine.

18. The method of claim 1 wherein said lysophospholipid is selected from the group consisting of LysoPC, LysoPA, LysoPS, LysoPE, LysoPI, and LysoPG lysophospholipids.

19. The method of claim 1 further comprising the step of determining the concentration of lysophospholipids having a particular degree of saturated or unsaturated fatty acids and/or fatty acid chain length.

20. The method of claim 1 further comprising the step of determining the concentration of lysophospholipids having a particular long chain alcohol.

21. The method of claim 1 wherein said sample is selected from the group consisting of plasma, serum, urine, saliva, ascites, cerebral spinal fluid and pleural fluid.

22. A method for detecting the presence of cancer in a sample of bodily fluid taken from a test subject, comprising the steps of:
(a) determining the concentration of a lysophospholipid in a sample of bodily fluid from said test subject;
(b) determining the concentration of the fatty acids of said lysophospholipid in the sample from said test subject; and
(c) comparing the values obtained in step a and b with values obtained from controls.

23. The method of claim 22 wherein said concentrations are normalized to the concentration of a compound selected from the group of other lysophospholipids, phospholipids, albumen and creatinine.

24. The method of claim 22 wherein said lysophospholipid is selected from the group consisting of LysoPC, LysoPA, LysoPS, LysoPE, LysoPI and LysoPG lysophospholipids.

25. The method of claim 22 where said cancer is a gynecological cancer.

26. The method of claim 1 or 22 wherein said concentration of lysophopholipid is taken at successive time intervals to establish a rate of change over time for the concentration of the lysophospholipid.

27. A method for monitoring the presence of cancer in a test subject over time, comprising:
a) determining the concentration of a lysophospholipid in a sample of bodily fluid taken from said test subject at a first time;
b) determining the concentration of the lysophospholipid in a sample of bodily fluid taken from the test subject at a later time; and
c) comparing the concentrations obtained in step a and b to determine whether there has been an increase or decrease in the concentration of the lysophospholipid in the sample taken from said test subject at the later time relative to the concentration of the lysophospholipid in a sample taken from the test subject at said first time,
whereby an increase in the concentration of the lysophospholipid in the sample from said later time indicates an increase in the number of viable tumor cells and a decrease indicates a decrease in the number of viable tumor cells.

28. The method of claim 27 wherein said tumor is a gynecological cancer selected from the group consisting of ovarian, fallopian tube, uterine, intraperitoneal carcinomatosis and cervical cancers.

29. The method of claim 27 wherein said step of determining the concentration of lysophospholipid comprises contacting said sample with an anti-lysophospholipid antibody.

30. The method of claim 27 further comprising the step of determining the fatty acid composition of said lysophospholipid and the concentration of said fatty acids of the lysophospholipid in the samples.

31. The method of claim 27 further comprising the step of determining the concentration of lysophospholipids having a particular long chain alcohol.

32. The method of claim 27 wherein said lysophospholipid is a sn-1 or sn-2 lysophospholipid having a glycerol backbone with a phosphate or derivatized phosphate at the sn-3 position and having a single fatty acid chain located at the sn-1 or sn-2 position linked by an acyl linkage and having a hydroxyl located at the other sn-1 or sn-2 position.

33. The method of claim 27 wherein said lysophospholipid is a sn-1 lysophospholipid having a glycerol backbone with a phosphate or derivatized phosphate at the sn-3 position and having a long chain alcohol located at the sn-1 position linked by an alkyl or alkenyl linkage and having a hydroxyl located at the sn-2 position.

34. The method of claim 27 wherein said lysophospholipid is selected from the group consisting of LysoPC, LysoPA, and LysoPS, LysoPE, LysoPI, and LysoPG lysophospholipids.

35. A diagnostic kit for detecting the concentration of lysophospholipids in a sample of bodily fluid taken from a test subject to detect cancer, said kit comprising reagents for measuring the concentrations of lysophospholipids in a sample of bodily fluid taken from a test subject.

36. The diagnostic kit of claim 35 wherein said reagents include an anti-lysophospholipid antibody.

37. The diagnostic kit of claim 35 further comprising a reagent for inhibiting production or hydrolysis of lysophospholipid in the sample during transport or storage.
